(19) United States
(12) Patent Application Publication
(10) Pub. No.: US 2023/0372167 A1
(43) Pub. Date: Nov. 23, 2023

(54) ADHESIVE FASTENER AND DIAPER HAVING SUCH AN ADHESIVE FASTENER

(71) Applicant: Gottlieb Binder GmbH & Co. KG, Holzgerlingen (DE)

(72) Inventors: Konstantinos POULAKIS, Hildrizhausen (DE); Lars HEEPE, Tübingen (DE); Clovis NKUIDJEU, Stuttgart (DE)

(21) Appl. No.: 18/030,543

(22) PCT Filed: Sep. 22, 2021
(86) PCT No.: PCT/EP2021/076072

§ 371 Date: Apr. 6, 2023

(30) Foreign Application Priority Data
Oct. 9, 2020 (DE) 10 2020 006 241.5

(51) Int. Cl. A61F 13/62; A44B 18/00; A61F 13/15

(52) U.S. Cl. CPC A61F 13/625; A44B 18/0065; A61F 13/15252

(57) ABSTRACT

1. Adhesive fastener and diaper having such an adhesive fastener. 2. An adhesive fastener, consisting of at least one plastic material, having a plurality of hook means which are integrally connected to a substrate (10), each in the form of a stalk part (14) which is provided with a head part (12) and which protrudes above the substrate (10), is characterized in that at least a portion of the plastic material used is biodegradable and/or is bio-based.

The invention relates to an adhesive fastener consisting of at least one plastic material having a plurality of hook means connected integrally to a substrate, each in the form of a stalk part which is provided with a head part and which protrudes above the substrate. The invention further relates to a diaper having at least one such adhesive fastener.

Diapers, such as baby or incontinence diapers, as they are used on the world market, are regularly designed as disposable diapers, which leads to considerable amounts of waste, the environmentally-friendly disposal of which must be regarded as problematic. Disposable diapers are nowadays offered on the market as so-called eco-friendly diapers in environmentally-friendly variants, and have a higher proportion of biodegradable components which are at least partially obtained from sustainable resources. These eco-friendly diapers are not disposed of in organic waste collection, even though the production material thereof is about 85% biodegradable; rather, they are disposed of via the standard waste collection—usually intended for combustion in heating power plants. This cannot exactly be seen as environmentally friendly.

The republished DE 10 2019 006 683 A1 also discloses a baby or incontinence diaper consisting of an outer shell and an absorption body connected to this shell, wherein, by means of a mechanical closure system acting between the outer shell and the absorption body—typically in the form of correspondingly interacting adhesive fasteners—the absorption body can be separated from the shell. This helps to relieve the environmental burden—in particular, if the absorption body is constructed from biodegradable pulp materials which can be mechanically separated from the other components, such as the outer shell of the diaper, and disposed of in the organic waste collection. The specific outer shell of the diaper—particularly if it is constructed of compostable plastic materials—can then be returned directly into the recycling system.

Such baby or incontinence diapers typically have laterally-arranged closure tabs with which the diaper can be closed in a user-friendly manner after use. For this purpose, so-called hook-and-loop fasteners are regularly used, consisting of a hook material on one side and loops or fibers which can be hooked therein in a releasable manner on the other side, and which can also be formed by the surface material of the diaper itself. Such a closure system, in addition to a method for manufacturing the same, is known from DE 196 46 318 A1 of the patent owner. For the mentioned adhesive fasteners, which are typically sewn or glued onto the closure tabs of a baby diaper, high-quality, temperature-resistant plastic materials are used—preferably polyamide, and in particular polyamide pellets or polyamide-containing mixtures of thermoplastics. However, such plastics, although they are functionally reliable and useful in the application, have the disadvantage that they are difficult to recycle due to the mentioned plastic material. In particular, this material cannot be easily degraded biologically. Their use in diapers accordingly also makes the environmentally-friendly disposal or preparation of the diaper as a whole more difficult, even if the rest of the production material is completely biodegradable.

Proceeding from this prior art, it is therefore the object of the invention to further improve the known adhesive fasteners in such a way that they can be disposed of in an environmentally-friendly manner, as required—which also includes areas of application for such an adhesive fastener such as, for example, the use in the field of diapers of any type.

Such an object is achieved by an adhesive fastener having the features of claim 1 in its entirety, and by a diaper having at least one such adhesive fastener according to the features of claim 7.

According to the characterizing part of claim 1, an essential feature of the adhesive fastener according to the invention is that at least a portion of the plastic material used is biodegradable and/or bio-based. Such adhesive fasteners are preferably used in a diaper whose production or use material is constructed to be at least about 85% biodegradable and/or made of bio-based materials.

Preferably, the biodegradable plastic material is polycaprolactone, polybutyrate, or polybutylene succinate. Even though the material polycaprolactone is based upon petroleum products, this plastic can be degraded biologically by the action of microorganisms, such as fungi or bacteria. Furthermore, the degradation can be accelerated by the use of enzymes, wherein the biological degradation takes place substantially by oxidation and hydrolysis processes to form the dissociation products of water, carbon dioxide, or methane, and biomass. Another biodegradable plastic material is polybutyrate—more precisely, polybutyrate adipate terephthalate. This plastic material can include more than 30% of a bio-based carbon component, and has technical properties similar to those of LDPE (low-density polyethylene). Another suitable agent for the adhesive fastener is polybutylene succinate, which is based upon fossil raw materials involving the chemical compounds from the group of linear aliphatic polyesters, but can also be constructed from renewable raw materials. The technical properties thereof are again comparable to those of LDPE or polypropylene.

Cellulose acetate can be used as a bio-based plastic material for the adhesive fastener; it belongs to the thermoplastic plastics and can be constructed from semi-synthetic fibers. Furthermore, bio-based polyethylene, which can be produced both petrochemically and from an alcohol, such as bioethanol, can be used. If polyethylene furanoate is used, it is a 100% recyclable plastic material. All the above-mentioned bio-based plastics can be produced at least in part on the basis of renewable raw materials, which makes their use particularly environmentally friendly.

It is particularly advantageous to produce the adhesive fastener from a biodegradable plastic material that is bio-based. For this purpose, polyactides, which are also known by the term, polylactic acids, can be used as plastic materials. Further examples are polyhydroxyalkanoate and thermoplastic elastomers, including thermoplastic styrene block copolymers (TPE-S). All plastic materials used allow the substrate of the adhesive fastener to be designed in the manner of a plastic film which has a low melting point, which enables an adhesive-free connection to other components by thermal joining, such as components of a diaper—in particular, in the form of the closure tabs thereof.

For the use of the adhesive fastener in a diaper, it is preferably provided that the film thickness of the substrate be between 0.30 and 0.36 mm at a melting temperature of the material of 70° C. Such an adhesive fastener can then, with a peel adhesion to a loop or ringlet material as a further adhesive fastener of a releasable adhesive closure, have average values of 2.34 N/cm².

Such a diaper equipped with these adhesive fasteners—particularly in the region of its closure tabs—makes possible the complete biological degradability of the entire used diaper.

It is understood that the individual plastic materials mentioned can also be mixed with one another to form a compound which can be provided with corresponding fillers for its stabilization.

The adhesive fastener according to the invention and the diaper having at least one such adhesive fastener are explained in more detail below with reference to the drawing. The figures show, in schematic illustrations which are not to scale:

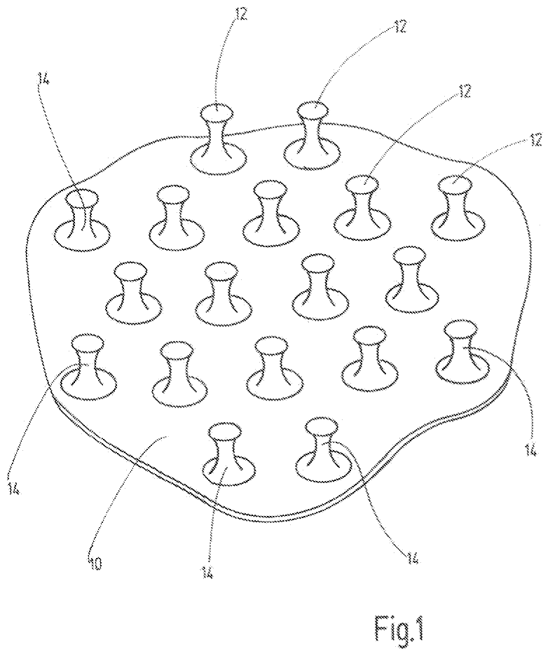

FIG. 1 a perspectival view of a portion of an adhesive fastener, and

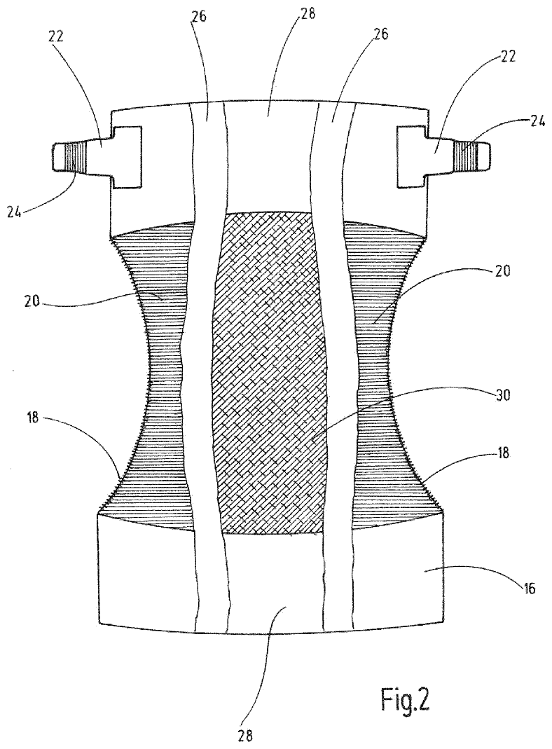

FIG. 2 a plan view of a diaper pressed flat in the plane of the drawing.

The adhesive fastener shown in part in FIG. 1 has a plurality of hook means which are connected integrally to a film-like substrate 10, and symmetrically structured hook means each in the form of a stalk part 14 connected to a head part 12. To produce this adhesive fastener, a method is used in which a moldable plastic material is fed into a shaping zone between a roller-like printing tool and a roller-like molding tool. The mentioned roller-like tools are driven in such a way that the substrate is formed in the shaping zone and is conveyed in a predeterminable transport direction. A sieve is fixed, as the shaping zone, on the molding tool, and has individual mold cavities, wherein, at least in a longitudinal section of each individual mold cavity, the opposing boundary walls have a continuous convex profile to form the stalk part of the respective stalk parts 14. The head parts 12 projecting outwards can be recalendered to form a single head plane, after they leave the molding tool. Furthermore, so as to create a resistant stalk part 14, it is rotationally symmetrical, as is the head part 12. Such a shaping production method is also referred to in technical terms as the “chill-roll method.”

In addition to the plastic materials already described above, a polyolefin-based plastic material has proven to be particularly advantageous, using additives as described by way of example in WO 2018/095905 A1. Furthermore, thermoplastically-processable, biodegradable molding compositions, containing completely biodegradable polyesteramides and thermoplastically-processed cellulose derivatives, are suitable, as described in EP 0780439 A2.

This enumeration of suitable plastic materials, which are biodegradable and/or bio-based, is not exhaustive.

FIG. 2 shows the outer shell 16 of a disposable diaper, in the form of a baby diaper or incontinence diaper for adults, opened flat. The outer shell 16 is formed with a taper along its two, mutually-opposite, longitudinal edges 18, so that leg openings are formed for a user. The two longitudinal regions 20 forming a kind of cuff are formed from an elastic material that is customary in diapers, which increases the wearing comfort for the user. On the upper end of the outer shell 16 as viewed in the viewing direction of FIG. 1, two flap-like closures or closure tabs 22 are attached in a conventional manner, with which the diaper can be fixed around a user and can be closed after use by rolling up. For this purpose, the two closure tabs 22, in the direction of their two free ends, have adhesive fasteners 24 with a closure material, as is shown in detail in FIG. 1. The adhesive fasteners 24, attached along the two longitudinal edges 18 on the head side of the diaper and fixed on the front side of the diaper in a so-called target area, and which enable a secure placement of the diaper on its user, are arranged like strips on the end of the closure tabs 22.

Along the two longitudinal edges 12, in the viewing direction of FIG. 2, two cover strips 26 extend from the top to the bottom, to the right and left of the center of the sheath 16, and accommodate an absorption body 30 between them while leaving two end regions 28 free. For this purpose, the cover strips 26 are fixedly connected to the sheath 10 in the direction of the two longitudinal edges 18, and can be lifted like a pocket in the direction of their two adjacent edge regions to be able to accommodate the absorption body 22 in the diaper in such a way that it overlaps at the edges and covers the diaper.

A suitable absorber material for the absorption body 22 is a fluid-permeable gauze sheath which comprises cellulose flakes inside the absorption body 30, serving to absorb fluid—which is typically the excretions of the diaper user. Further details on the construction of such a diaper are found in the republished patent DE 10 2019 006 683 A1.

The substrate 10, which is formed in the manner of a film from the given plastic material, has a low melting point, which enables an adhesive-free connection to other components by thermal joining—such as components of a diaper in the form of the two closure tabs 22. The melting temperature of the other component in the form of the respective closure tabs 22 on the diaper is preferably greater than the melting temperature of the substrate 10 of the adhesive fastener made of the film material.

As further indicated in FIG. 1, the film thickness of the substrate 10 is between 0.30 mm and 0.36 mm, with a melting temperature of about 70° C. This results in a peel adhesion with respect to a loop or ringlet material as a further adhesive fastener (not shown) of a detachable adhesive closure or an upper side of the diaper of 2.34 N/cm² on average. Instead of the further adhesive fastener, the closure system can also be formed by the loop or ringlet material of the surface material of the diaper. Overall, a diaper constructed in this way can be largely biologically degraded after use and/or made of bio-based materials.

A verification of the elongation at break and/or the tear strength has resulted in the following properties. The measurements were carried out along the entire width of the film-like adhesive fastener (hook-and-loop film) by means of a so-called Zwicki from the Zwick/Roell company. The strips measured in this way had a width of 20 mm with a length of 200 mm. If polylactide (PLA) material is used for the adhesive fastener as a whole, as can be obtained on the market under the trade name lnzea, elongations at break of >100%, and a tear strength of >40 N, are obtained; for the product ArcBio, as a PLA material, an elongation at break of >15% and a tear strength of >90 N are obtained.

For measurements of the free surface energy using two liquids (water and diiodomethanes) on the rear side of the adhesive fastener (right, center, and left) using a drop shape analyzer from the Krüss company, for lnzea, a polar fraction of >0.01 mJ/m² with a dispersed fraction of >39 mJ/m² is obtained. For the ArcBio material, the polar fraction is >6 mJ/m² with a dispersed fraction of >39 mJ/m².

The measurement of the closure thickness was performed by means of a DM 2000 thickness measuring device from Wolf measurement technology, demonstrating, for example, a thickness of approximately 0.360 mm for the lnzea adhesive fastener, and a thickness of approximately 0.343 mm for the ArcBio.

Measurement of the heat distortion resistance according to DIN EN ISO 75 [HDT 0.45 MPA (B)] demonstrated an HDT value of approximately 50° C. for lnzea and an HDT value of approximately 96° C. for ArcBio.

1. Adhesive fastener, consisting of at least one plastic material, having a plurality of hook means integrally connected to a substrate (10), each in the form of a stalk part (14) which is provided with a head part (12) and which protrudes above the substrate (10), characterized in that at least a portion of the plastic material used is biodegradable and/or bio-based.

2. Adhesive fastener according to claim 1, characterized in that the biodegradable plastic material is
polycaprolactone (PCL),
polybutyrate (PBAT), or
polybutylene succinate (PBS).

3. Adhesive fastener according to claim 1, characterized in that the bio-based plastic material is
cellulose acetate (CA),
bio-based polyethylene (Bio-PE), or
polyethylene furanoate (PEF), or
bio-based polyamide (Bio-PA), or
bio-based polyethylene terephthalate (Bio-PET).

4. Adhesive fastener according to claim 1, characterized in that the biodegradable plastic material is bio-based, consisting of
polylactide (PLA),
polyhydroxyalkanoate (PHA), or
thermoplastic elastomers (TPS), or
starch

5. Adhesive fastener according to claim 1, characterized in that the substrate (10) is formed in the manner of a film of plastic material which has a low melting point, which enables an adhesive-free connection to other components by thermal joining, such as components of a diaper.

6. Adhesive fastener according to claim 1, characterized in that the melting temperature of the other component, such as a closure tab (22) on the diaper, is greater than the melting temperature of the substrate (10) made of the film material.

7. Diaper, consisting of a production material that is at least about 85% biodegradable and/or constructed from bio-based materials, and has at least one adhesive fastener according to claim 1.

8. Diaper according to claim 7, characterized in that it is provided with closure tabs which have at least one adhesive fastener.

9. Diaper according to claim 8, characterized in that an adhesive fastener is permanently bonded to the closure tabs (22) by adhesive-free, thermal joining.
